(19) United States
(12) Patent Application Publication
(10) Pub. No.: US 2025/0099346 A1
(43) Pub. Date: Mar. 27, 2025

(54) W/O/W EMULSION CONTAINING INOSITOL AND METHOD FOR PRODUCING SAME

(71) Applicant: SUNTORY HOLDINGS LIMITED, Osaka-shi, Osaka (JP)

(72) Inventors: Tomoyuki Doi, Soraku-gun (JP); Tatsuo Matsuoka, Soraku-gun (JP)

(73) Assignee: SUNTORY HOLDINGS LIMITED, Osaka-shi, Osaka (JP)

(21) Appl. No.: 18/730,561

(22) PCT Filed: Jan. 26, 2023
(86) PCT No.: PCT/JP2023/002340

§ 371 Date: Jul. 19, 2024

(30) Foreign Application Priority Data
Jan. 27, 2022 (JP) 2022-011297

(51) Int. Cl. A61K 8/06; A61K 8/34; A61K 8/894; A61K 8/92; A61Q 19/00

(52) U.S. Cl. CPC A61K 8/066; A61K 8/345; A61K 8/894; A61K 8/92; A61Q 19/007; A61K 2800/5422; A61K 2800/805

(57) ABSTRACT

The present invention aims to provide a W/O/W emulsion containing inositol and having high temporal stability and a method of producing the W/O/W emulsion. The present invention relates to, for example, a W/O/W emulsion containing an inner aqueous phase, an oil phase, and an outer aqueous phase, the W/O/W emulsion containing inositol in the inner aqueous phase.

### TECHNICAL FIELD

The present invention relates to a W/O/W emulsion and a method of producing the W/O/W emulsion. More specifically, the present invention relates to a W/O/W emulsion containing inositol and having high temporal stability and a method of producing the W/O/W emulsion.

### BACKGROUND ART

A W/O/W (water-in-oil-in-water) emulsion is a multiphase emulsion in which a water-in-oil (W/O) emulsion is further emulsified and dispersed in an aqueous phase (outer aqueous phase). Such W/O/W emulsions are known as a formulation technique that can improve the percutaneous absorbability of active ingredients as compared to other emulsion types (Non-Patent Literature 1) and are expected to be applied to various applications including cosmetics, foods, pharmaceuticals, and the like.

Meanwhile, inositol is known to have actions such as a moisturizing action and a sebum secretion regulating action. Patent Literature 1 discloses a pharmaceutical composition for external use, which contains inositol and which is in an oil-in-water emulsified form.

### CITATION LIST

### Patent Literature

Patent Literature 1: JP 2019-218319 A

### Non-Patent Literature

Non-Patent Literature 1: Fragrance Journal, 1989, Vol. 4, pp. 88-93

### SUMMARY OF INVENTION

### Technical Problem

According to Patent Literature 1, adding inositol to an aqueous phase of a cream in an oil-in-water emulsified form improved the emulsion stability of the cream. However, Patent Literature 1 is silent about a W/O/W emulsion containing inositol. The aqueous phase of an oil-in-water emulsion corresponds to the outer aqueous phase of a W/O/W emulsion.

As described above, inositol has actions such as a moisturizing action and a sebum secretion regulating action, so that a W/O/W emulsion containing inositol is useful, for example, in the field of cosmetics. Thus, the present inventors studied W/O/W emulsions containing inositol. Cosmetics are required to have high temporal stability because they are sometimes exposed to extremely harsh conditions in the stages of distribution and consumption by users. However, it was found that adding inositol to a W/O/W emulsion decreases the W/O/W emulsion stability and easily causes separation (phase separation) in some cases. Once a W/O/W emulsion is separated, the original emulsion cannot be restored.

The present invention aims to provide a W/O/W emulsion containing inositol and having high temporal stability and a method of producing the W/O/W emulsion.

### Solution to Problem

As a result of extensive studies to obtain a W/O/W emulsion containing inositol and having high temporal stability, the present inventors found that a W/O/W emulsion is stabilized when the W/O/W emulsion contains inositol in its inner aqueous phase.

Specifically, the present invention relates, but is not limited, to a W/O/W emulsion and a method of producing the W/O/W emulsion, which are defined below.
(1) A W/O/W emulsion containing an inner aqueous phase, an oil phase, and an outer aqueous phase, the W/O/W emulsion containing inositol in the inner aqueous phase.
(2) The W/O/W emulsion according to (1) above, wherein the W/O/W emulsion contains no inositol in the outer aqueous phase.
(3) The W/O/W emulsion according to (1) or (2) above, wherein the W/O/W emulsion has an inositol content of 0.01 to 1.4 wt %.
(4) The W/O/W emulsion according to any one of (1) to (3) above, wherein the W/O/W emulsion contains a solid oil component and a silicone surfactant in the oil phase, and a water-soluble polymer in the outer aqueous phase.
(5) The W/O/W emulsion according to (4) above, wherein the silicone surfactant has an HLB value of 7 or less.
(6) The W/O/W emulsion according to (4) or (5) above, wherein the silicone surfactant is a silicone surfactant having a polyether group and an alkyl group.
(7) The W/O/W emulsion according to (6) above, wherein the silicone surfactant having a polyether group and an alkyl group is cetyl PEG/PPG-10/1 dimethicone or lauryl PEG-9 polydimethylsiloxyethyl dimethicone.
(8) The W/O/W emulsion according to any one of (4) to (7) above, wherein the W/O/W emulsion has a solid oil component content of 0.5 wt % or more.
(9) The W/O/W emulsion according to any one of (4) to (8) above, wherein the solid oil component is a wax.
(10) The W/O/W emulsion according to any one of (4) to (9) above, wherein the water-soluble polymer is an acrylic acid/alkyl methacrylate copolymer.
(11) The W/O/W emulsion according to any one of (1) to (10) above, further containing a metal salt in the inner aqueous phase.
(12) The W/O/W emulsion according to any one of (1) to (11) above, wherein a weight ratio of the inner aqueous phase to the oil phase is 10/90 to 90/10.
(13) The W/O/W emulsion according to any one of (1) to (12) above, wherein the W/O/W emulsion is an agent for external use.
(14) The W/O/W emulsion according to any one of (1) to (13) above, wherein the W/O/W emulsion is a skin agent for external use.
(15) The W/O/W emulsion according to any one of (1) to (14) above, wherein the W/O/W emulsion is a cream or a milky lotion.
(16) A method of producing a W/O/W emulsion, including: a step of mixing and emulsifying an inner aqueous phase containing inositol and an oil phase to prepare a W/O emulsion; and a step of mixing and emulsifying the prepared W/O emulsion and an outer aqueous phase.
(17) The method of producing a W/O/W emulsion according to (16) above, wherein the outer aqueous phase contains no inositol.
(18) The method of producing a W/O/W emulsion according to (16) or (17) above, wherein the oil phase contains a solid oil component and a silicone surfactant, and the outer aqueous phase contains a water-soluble polymer.
(19) The method of producing a W/O/W emulsion according to any one of (16) to (18) above, wherein the method includes mixing the inner aqueous phase and the oil phase at a weight ratio of 10/90 to 90/10.

### Advantageous Effects of Invention

The present invention can provide a W/O/W emulsion containing inositol and having high temporal stability and a method of producing the W/O/W emulsion. The W/O/W emulsion of the present invention is useful, for example, as a skin agent for external use such as a cosmetic.

### DESCRIPTION OF EMBODIMENTS

Hereinafter, embodiments of the present invention are described as examples.

(W/O/W emulsion)

The W/O/W emulsion of the present invention is an W/O/W emulsion containing an inner aqueous phase, an oil phase, and an outer aqueous phase, the W/O/W emulsion containing inositol in the inner aqueous phase.

The W/O/W emulsion of the present invention is a W/O/W emulsion, which usually contains an inner aqueous phase, an oil phase in which the inner aqueous phase is dispersed (emulsified), and an outer aqueous phase in which the oil phase is dispersed (emulsified). The W/O/W emulsion of the present invention may consist of the inner aqueous phase, the oil phase, and the outer aqueous phase. 30 In a W/O/W emulsion, a W/O emulsion containing an inner aqueous phase (inner aqueous phase droplets) dispersed (emulsified) in an oil phase is dispersed (emulsified) in an outer aqueous phase.

The W/O/W emulsion of the present invention has high temporal stability and is not prone to separation so that it is useful in the field of cosmetics and the like.

The terms “inner aqueous phase”, “oil phase”, and “outer aqueous phase” used in the present invention may refer to the respective phases of the W/O/W emulsion or respective solutions containing respective phase components for use in producing the W/O/W emulsion.

### (Inner Aqueous Phase Component)

The W/O/W emulsion of the present invention contains inositol in the inner aqueous phase. When the W/O/W emulsion contains inositol in the inner aqueous phase, the W/O/W emulsion has high temporal stability.

Inositol (1, 2, 3, 4,5,6-cyclohexanehexol) is a compound having a structure in which one hydrogen atom on each carbon of cyclohexane is substituted with a hydroxyl group. Depending on combinations of steric configurations of hydroxy groups, inositol exists as nine stereoisomers including myo-inositol, allo-inositol, D-chiro-inositol, L-chiro-inositol, cis-inositol, epi-inositol, muco-inositol, neo-inositol, and scyllo-inositol.

In the present invention, inositol may be one of the nine stereoisomers or a combination of two or more thereof. In the present invention, inositol is preferably myo-inositol (cis-1, 2, 3, 5-trans-4,6-cyclohexanehexol).

The inositol content of the W/O/W emulsion of the present invention may be 0.001 wt % or more, preferably 0.01 wt % or more, more preferably 0.05 wt % or more, still more preferably 0.08 wt % or more, particularly preferably 0.1 wt % or more. The inositol content of the W/O/W emulsion may be 0.2 wt % or more, 0.5 wt % or more, or 1 wt % or more. The inositol content of the W/O/W emulsion is preferably 2 wt % or less, more preferably 1.5 wt % or less, still more preferably 1.4 wt % or less, yet still more preferably 1 wt % or less, particularly preferably 0.5 wt % or less, most preferably 0.2 wt % or less. The inositol content of the W/O/W emulsion may be 0.1 wt % or less or 0.08 wt % or less. The W/O/W emulsion having an inositol content in the above range has a higher temporal stability. Thus, the W/O/W emulsion is not prone to separation even when stored for a long time, exhibiting long-term storage stability.

In one embodiment, the inositol content of the W/O/W emulsion may be 0.001 to 2 wt %, preferably 0.01 to 1.5 wt %, more preferably 0.01 to 1.4 wt %, still more preferably 0.05 to 1.4 wt %, yet still more preferably 0.08 to 1.4 wt %, further preferably 0.1 to 1.4 wt %, still further preferably 0.1 to 1 wt %, particularly preferably 0.1 to 0.5 wt %, most preferably 0.1 to 0.2 wt %.

Preferably, the W/O/W emulsion of the present invention contains the above amount of inositol in the inner aqueous phase. Inositol in the inner aqueous phase can be measured by a known method (e.g., high performance liquid chromatography (HPLC)) after separating the inner aqueous phase, the oil phase, and the outer aqueous phase of the W/O/W emulsion using a known method (e.g., centrifugation).

Inositol is known to have actions such as a moisturizing action and a sebum secretion regulating action. The W/O/W emulsion of the present invention can be used, for example, to obtain actions derived from inositol.

Preferably, the W/O/W emulsion of the present invention further contains a metal salt in the inner aqueous phase. The W/O/W emulsion containing a metal salt as an inner aqueous phase component has a higher temporal stability. Any metal salt may be used. Preferred examples include magnesium sulfate, sodium citrate, amino acid salts such as sodium glutamate, sodium lactate, and sodium chloride, with magnesium sulfate being more preferred. One or more metal salts can be used. When the W/O/W emulsion contains a metal salt, the metal salt content of the W/O/W emulsion is not limited but may be, for example, 0.001 to 10 wt %, preferably 0.01 to 5 wt %, more preferably 0.1 to 2 wt %.

A water-soluble active ingredient other than inositol may be contained in the inner aqueous phase, as long as the effects of the present invention are not impaired. One or more water-soluble active ingredients other than inositol may be used. Examples of the water-soluble active ingredients other than inositol include those known as skin-lightening components such as ascorbic acid derivatives (vitamin C derivatives), arbutin, Chamomile ET, tranexamic acid, placenta extract, lucinol, and hydroquinone. Examples also include plant extracts and other beautifying components, which are mentioned later. When the W/O/W emulsion contains a water-soluble active ingredient other than inositol, the water-soluble active ingredient content of the W/O/W emulsion is not limited but may be, for example, 0.0001 to 10 wt %, preferably 0.001 to 5 wt %, more preferably 0.005 to 5 wt %, still more preferably 0.005 to 2 wt %. In one embodiment, the water-soluble active ingredient content (other than inositol) of the W/O/W emulsion is also preferably 0.001 to 2 wt %.

### (Oil Phase Component)

An oil component is contained in the oil phase. The oil component may be a liquid oil component or a solid oil component. One or more oil components may be used.

Usually, a liquid oil component is contained in the oil phase. Any liquid oil component that is a liquid at room temperature may be used. Examples of the liquid oil component include natural animal and vegetable fats and oils, hydrocarbon oils, ester oils, silicone oils, higher alcohols, and higher fatty acids. One or more of these can be used.

Examples of the natural animal and vegetable oils include avocado oil, linseed oil, almond oil, olive oil, cacao oil, tung oil, wheat germ oil, sesame oil, rice germ oil, rice bran oil, safflower oil, soybean oil, evening primrose oil, camellia oil, corn oil, rapeseed oil, persic oil, palm oil, palm kernel oil, castor oil, sunflower oil, grape oil, jojoba oil, macadamia nut oil, mink oil, cottonseed oil, coconut oil, peanut oil, and rosehip oil.

Examples of the hydrocarbon oils include α-olefin oligomer, squalane, squalene, and liquid paraffin.

Examples of the ester oils include diisobutyl adipate, 2-hexyldecyl adipate, di-2-heptylundecyl adipate, isostearyl isostearate, trimethylolpropane triisostearate, cetyl 2-ethylhexanoate, neopentyl glycol di-2-ethylhexanoate, trimethylolpropane tri-2-ethylhexanoate, pentaerythritol tetra-2-ethylhexanoate, cetyl octoate, oleyl oleate, octyldodecyl oleate, decyl oleate, neopentyl glycol dicaprate, 2-ethylhexyl succinate, isocetyl stearate, butyl stearate, diisopropyl sebacate, cetyl lactate, tetradecyl lactate, isopropyl myristate, octyldodecyl myristate, cetyl myristate, myristyl myristate, octyl palmitate, 2-ethylhexyl palmitate, 2-hexyldecyl palmitate, 2-heptylundecyl palmitate, phytosteryl oleate, diisostearyl malate, p-methoxycinnamate ester, and pentaerythritol tetrarosinate.

Examples of the silicone oils include higher alkoxy-modified silicones, alkyl-modified silicones, higher fatty acid ester-modified silicones, such as dimethylpolysiloxane, highly polymerized methylpolysiloxane, methylphenylpolysiloxane, methylhydrogenpolysiloxane, octamethylcyclotetrasiloxane, octamethylcyclopentasiloxane, decamethylcyclohexasiloxane, and stearoxy silicone.

Examples of the higher alcohols include octyldodecanol, isostearyl alcohol, and oleyl alcohol.

Examples of the higher fatty acids include isostearic acid, oleic acid, linoleic acid, linolenic acid.

In one embodiment, the liquid oil component is preferably a hydrocarbon oil. In one embodiment, the liquid oil component is more preferably squalane.

Preferably, the W/O/W emulsion of the present invention contains a solid oil component and a silicone surfactant in the oil phase. The W/O/W emulsion containing a solid oil component and a silicone surfactant in the oil phase has a higher temporal stability. Preferably, the W/O/W emulsion contains no inositol in the oil phase.

The presence of a solid oil component as an oil phase component can increase the viscosity of the oil phase and reduce the fluidity of the inner aqueous phase. This can enhance the W/O/W emulsion stability. The solid oil component is not limited and can be selected from solid oils and fats, waxes, hydrocarbons, higher fatty acids, higher alcohols, esters, silicones, and the like, with solid fats and oils and waxes being particularly preferred. Examples of the solid oils and fats include cocoa butter, coconut oil, palm oil, palm kernel oil, hydrogenated oil, hydrogenated castor oil, Japan wax, and shea butter. Examples of the waxes include microcrystalline wax, beeswax, carnauba wax, candelilla wax, and lanoline. In particular, the solid oil component is preferably a wax, more preferably microcrystalline wax.

When the W/O/W emulsion contains a solid oil component, the solid oil component content is not limited. For example, the solid oil component content of the W/O/W emulsion is preferably 0.3 wt % or more, more preferably 0.5 wt % or more, and is preferably 10 wt % or less, more preferably 5 wt % or less. In one embodiment, the solid oil component content of the W/O/W emulsion is preferably 0.3 to 10 wt %, more preferably 0.5 to 5 wt. When a wax (preferably, microcrystalline wax) is used, the wax content of the W/O/W emulsion is preferably 0.4 wt % or more, more preferably 0.5 wt % or more, and is preferably 10 wt % or less, more preferably 5 wt % or less. In one embodiment, when the W/O/W emulsion contains a wax, the wax content of the W/O/W emulsion is preferably 0.4 to 10 wt %, more preferably 0.5 to 5 wt %.

The silicone surfactant is a surfactant having a structure in which a hydrophilic substituent is introduced into silicone, and various surfactants suitable for different applications can be obtained by changing the type of the hydrophilic substituent. In the present invention, any surfactant may be used as long as it is a silicone surfactant. For example, a polyether-modified silicone surfactant in which a polyether is used as the substituent is preferred, and a silicone surfactant having a polyether group and an alkyl group is more preferred.

The HLB value of the silicone surfactant is preferably 7 or less, more preferably 6 or less, still more preferably 2.5 to 5.5. When the HLB value of the silicone surfactant is in the above range, the resulting emulsion has a higher temporal stability.

Examples of the surfactant having a polyether group include PEG-11 methyl ether dimethicone, PEG/PPG-20/22 butyl ether dimethicone, PEG-9 dimethicone, PEG-3 dimethicone, PEG-9 methyl ether dimethicone, PEG-10 dimethicone, PEG-32 methyl ether dimethicone, polysilicone-13, PEG-9 polydimethylsiloxyethyl dimethicone, PEG/PPG-19/19 dimethicone, dimethicone/PEG-10/15 crosspolymer, cetyl PEG/PPG-10/1 dimethicone (HLB 5.0), and lauryl PEG-9 polydimethylsiloxyethyl dimethicone (HLB 3.0). Examples of the silicone surfactant having a polyether group and an alkyl group include lauryl or cetyl dimethicone copolyols. Particularly preferred examples include cetyl PEG/PPG-10/1 dimethicone and lauryl PEG-9 polydimethylsiloxyethyl dimethicone. One of the silicone surfactants can be used alone or two or more thereof can be used in combination.

When the W/O/W emulsion contains a silicone surfactant, the silicone surfactant content of the W/O/W emulsion may be, for example, 0.1 to 15.0 wt %, preferably 0.5 to 12.5 wt %, more preferably 1.0 to 10.0 wt %.

An oil-soluble active ingredient of any type may be optionally contained as an oil phase component. Further adding an oil-soluble active ingredient enables production of a cosmetic having a function derived from inositol and a function derived from the oil soluble active ingredient at the same time. The W/O/W emulsion of the present invention can contain, as oil-soluble active ingredients, the following plant extracts and other beautifying components, which are described as examples.

For example, a tocopherol derivative (vitamin E derivative) when contained in the oil phase can further add a blood circulation promoting action, an antioxidant action, and a skin softening effect to the W/O/W emulsion. When the W/O/W emulsion contains an oil-soluble active ingredient, the oil-soluble active ingredient content of the W/O/W emulsion may be, for example, 0.0001 to 10 wts, preferably 0.0005 to 5 wt %, more preferably 0.001 to 2 wt %.

### (Outer Aqueous Phase Component)

In the present invention, inositol may be contained in the outer aqueous phase but is preferably absent therein because the W/O/W emulsion has a higher temporal stability when the amount of inositol in the outer aqueous phase is smaller. The amount of inositol contained in the outer aqueous phase is preferably 0.5 wt % or less, more preferably 0.3 wt % or less, still more preferably 0.1 wt % or less in the W/O/W emulsion.

In the present invention, preferably, the W/O/W emulsion contains a water-soluble polymer in the outer aqueous phase. The W/O/W emulsion containing a water-soluble polymer in the outer aqueous phase has a higher temporal stability. The water-soluble polymer can be a water-soluble polymer known as a thickening agent. The water-soluble polymer is effective in preventing flocculation of W/O emulsion droplets because the water-soluble polymer increases the viscosity of the outer aqueous phase to reduce the flowability of the W/O emulsion.

One preferred embodiment of the W/O/W emulsion of the present invention is a W/O/W emulsion containing inositol in an inner aqueous phase, a solid oil component and a silicone surfactant in an oil phase, and a water-soluble polymer in an outer aqueous phase. Preferably, the W/O/W emulsion contains no inositol in the outer aqueous phase.

The water-soluble polymer for use in the present invention is not limited, and any known water-soluble polymer can be used. Examples include natural water-soluble polymers, semi-synthetic water-soluble polymers, and synthetic water-soluble polymers. Examples of the natural water-soluble polymers include plant-based polymers such as gum Arabic, guar gum, carrageenan, pectin, agar, quince (Cydonia oblonga) seed, and starches (e.g., rice, corn, potato, wheat); microbial polymers such as xanthan gum, dextran, and pullulan; and animal-based polymers such as collagen, casein, albumin, and gelatin. Examples of the semi-synthetic water-soluble polymers include starch polymers such as carboxymethyl starch and methylhydroxypropyl starch; cellulosic polymers such as methylcellulose, nitrocellulose, ethylcellulose, hydroxyethylcellulose, hydroxypropylcellulose, sodium carboxymethylcellulose (CMC), crystalline cellulose, and cellulose powder; and alginic acid polymers such as sodium alginate and propylene glycol esters of alginic acid. Examples of the synthetic water-soluble polymers include vinyl polymers such as polyvinyl alcohols, polyvinyl methyl ethers, and carboxyvinyl polymers (carbomers); copolymer-based polymers such as polyoxyethylene/polyoxypropylene copolymer; acrylic polymers such as sodium polyacrylate, polyacrylamides, and acrylic acid/alkyl methacrylate copolymers; polyethyleneimine; and cationic polymers. One water-soluble polymer may be used alone, or two or more water-soluble polymers may be used in combination. The water-soluble polymer is preferably an acrylic polymer, more preferably an acrylic acid/alkyl methacrylate copolymer.

Preferably, the W/O/W emulsion of the present invention contains an acrylic acid/alkyl methacrylate copolymer. When the W/O/W emulsion contains a water-soluble polymer, the water-soluble polymer content of the W/O/W emulsion may be, for example, 0.001 to 10 wts, preferably 0.005 to 5 wt %, more preferably 0.01 to 2 wt %.

Any other components may be present in the outer aqueous phase, and any component known to contribute to the emulsion stability can be contained. For example, a polyoxyethylene surfactant can be further contained. Any polyoxyethylene surfactant, such as polyoxyethylene sorbitan monooleate, can be used. When the W/O/W emulsion contains a polyoxyethylene surfactant, the polyoxyethylene surfactant content of the W/O/W emulsion may be, for example, 0.01 to 20 wt %, preferably 0.1 to 10 wt %, more preferably 1 to 5 wt %.

### (Other Components)

The W/O/W emulsion of the present invention may contain, as appropriate, various components generally used in cosmetics, quasi-pharmaceuticals, pharmaceuticals, perfumery, and the like. Examples include water, emulsifiers, humectants, thickening agents, active ingredients, antiseptic agents, pigments, powders, pH adjustors, ultraviolet absorbers, antioxidants, and fragrances.

As described above, the W/O/W emulsion of the present invention can contain both a water-soluble and oil-soluble active ingredient.

Examples of the active ingredient include collagen, hyaluronic acid, elastin, proteoglycan, ceramide, placenta, astaxanthin, polyphenols, yeast, amino acids, and peptides (e.g., acetyl hexapeptide-8).

A plant extract can also be added as an active ingredient. Examples include Persicaria tinctoria(Persicaria tinctoria leaf), Vitis vinifera, Rehmannia glutinosa (Rehmannia Root), Cannabis sativa (Mashiningan), Angelica keiskei, Phaseolus angularis, Malpighia emarginata, Uncaria gambir, Persea americana, Linum usitatissimum, Hydrangea macrophylla var. thunbergii, Hydrangea macrophylla var. thunbergii leaf, Gynostemma pentaphyllum, Althaea, Arnica montana, Aloe, Aloe vera, Prunus armeniaca var. ansu/Prunus armeniaca var. typica, Fragaria, Ficus carica or its leaf, Ginkgo biloba (Ginkgo biloba leaf/nut), Oryza sativa seed or seed coat, Urtica thunbergiana, Cananga odorata, Curcuma longa, Malva sylvestris, Althaea officinalis, Prunella vulgaris subsp. asiatica, Prunus mume (Prunus mume fruit) or its pulp, oolong, Citrus unshiu (Citrus unshiu peel), Eleutherococcus senticosus, Rubus idaeus subsp. melanolasius, Flammulina velutipes, Senna obtusifolia (juemingzi), Sambucus nigra fruit, Styphnolobium japonicum (sophora flower, sophora bud), Astragalus root/Astragalus membranaceus, Coptis japonica, Plantago asiatica (Plantago asiatica seed/leaf), Crataegus pinnatifida/Crataegus cuneata, Agave filifera var. compacta, Hordeum vulgare, Abelmoschus esculentus fruit, Panax ginseng/Panax (ginseng), Quercus (oak), Hypericum erectum/Hypericum perforatum var. angustifolium/Hypericum perforatum, Lamium album var. barbatum (Dipsacus japonicus), Juglans mandshurica var. sachalinensis, Lilium lancifolium/Lilium japonicum/Lilium brownii var. corchesteri (Lilium lancifolium), Ononis, Ulmus laciniata, Fragaria ananassa, Nasturtium officinale (cresson), Petroselium crispum, Apium graveolens var. dulce, Olea europaea, Origanum vulgare, Citrus sinensis fruit or peel, Rehmannia glutinosa var. hueichingensis (steamed and dried Rehmannia root), Theobroma cacao seed, Diospyros kaki or its leaf (dried calyx of Diospyros kaki), Curcuma zedoaria Roscoe, Quercus dentata (Quercus dentata tree/leaf), Betula or Betula pendula, Cucurbita, Typha latifolia, Matricaria recutita/Anthemis nobilis, Paullinia cupana seed, Chaenomeles sinensis, Garcinia cambogia, Salix gracilistyla, Trametes versicolor, Artemisia capillaris, Glycyrrhiza uralensis, Agave cantala, Tussilago farfara, Canna generalis, Rubus, Actinidia deliciosa fruit, Chrysanthemum morifolium (Chrysanthemum morifolium flower), Catalpa ovata, Rumex japonicus, Citrus aurantium, Hedera rhombea, Aloe arborescens, Satureja hortensis, Cinchona, Cinchona succirubra (Cinchona, Cinchona pubescens), Brassica oleracea var. capitata, unripe fruit of Brassica oleracea var. capitata, Cucumis sativus, Fortunella fruit, Piper betle, Psidium guajava fruit, Parthenium argentatum, Quercus infectoria (gallnut), Aleurites moluccanus, Lycium chinense (Lycium chinense fruit/leaf/root bark), Pueraria lobata (Pueraria lobata root), Cinnamomum camphora, Ribes uva-crispa fruit, Gardenia jasminoides, Sasa veitchii, Verbena officinalis, Sophora flavescens, Vaccinium macrocarpon fruit, Castanea crenata or its astringent skin, Citrus paradisi, Isodon trichocarpus, Alpinia zerumbet (Alpinia zerumbet leaf), Laurus nobilis, Gentiana lutea, Gentiana lutea rhizome/root, Camellia sinensis, Scutellaria baicalensis (Scutellaria root), Vaccinium vitis-idaea, Piper nigrum, Coffea arabica bean, Arctium lappa (Arctium seed), Sesamum indicum, Scrophulariaceae Juss., Schisandra chinensis, Triticum aestivum, Oryza sativa/Oryza sativa bran (red/white bran)/Oryza sativa oil/hydrolyzed Oryza sativa extract, Amorphophallus konjac, leaf/flower/fruit/bark of Prunus (Prunus Iannesiana var. speciosa, Prunus jamasakura, Prunus sargentii, Prunus pendula f. ascendens, Prunus incisa, Prunus maximowiczii, Prunus yedoensis, Prunus nipponica, Prunus verecunda, Prunus apetala, Prunus subhirtella, Prunus lannesiana, and Prunus kanzakura), Prunus avium, Punica granatum, Ipomoea batatas, Saccharum officinarum, Beta vulgaris ssp. vulgaris, Salvia (Salvia officinalis), Zanthoxylum, Butyrospermum parkii (shea butter tree), Lentinula edodes, Aster tataricus, Perilla frutescens/Perilla frutescens var. crispa f. viridis/Perilla frutescens var. crispa f. crispa/Perilla frutescens var. crispa ‘Discolor’ (Perilla frutescens leaf/seed), Tilia japonica, Lyophyllum decastes, Hypsizygus marmoreus, Hypsizygus ulmarius, Filipendula, Solanum tuberosum, Paeonia lactiflora, Paeonia lactiflora root, Zingiber officinale, Acous calamus (Acous calamus root), Iris florentina, flower of Cymbararia muralis f. alba, Citrullus lanatus, Lonicera japonica (Japanese honeysuckle), Stevia rebaudiana, Fragaria fruit, Equisetum arvense, Stevia rebaudiana, Prunus salicina fruit, Pinus sylvestris cone, Larix decidua, Hedera helix, Juglans regia, Crataegus oxyacantha, Taraxacum officinale, Aesculus hippocastanum (horse-chestnut), Pyrus communis (Pyrus communis fruit/branch), Filipendula ulmaria, Achillea millefolium (milfoil), Rosa centifolia, Mentha piperita/Melissa officinalis, Malva sylvestris var. mauritiana, Oenanthe javanica, Apium graveolens, Cnidium officinale, Swertia japonica, Fagopyrum esculentum seed, Rheum palmatum, Raphanus sativus, Glycine max, Citrus aurantium (Citrus aurantium peel/fruit), Thymus vulgaris (Thymus quinquecostatus, wild thyme, Thymus mongolicus Ronniger), Rosa Damascena (Rosa Damascena flower), Allium cepa, Magnolia salicifolia (Magnolia kobus), Aralia elata or its root bark, Salvia miltiorrhiza, Taraxacum hondoense or Taraxacum albidum/Taraxacum mongolicum, Prunus avium fruit, Panax ginseng (ginseng), Oenothera tetraptera, Camellia japonica, Centella asiatica, Commelina communis, Vigna umbellata, Polygonum multiflorum, Rubus caesius fruit, Benincasa hispida (Benincasa hispida seed), Capsicum annuum, Angelica acutiloba, Calendula officinalis (marigold), Zea mays or Zea mays silk, Houttuynia cordata, Carapichea ipecacuanha, Eucommia ulmoides (Eucommia ulmoides leaf), Solanum lycopersicum, Potentilla tormentilla, Dioscorea batatas/Dioscorea japonica, Citrus natsudaidai, Zizyphus jujuba var. inermis, Arachis hypogaea, Nandina domestica (Nandina domestica fruit), Myristica, Cinnamomum loureiri/Cinnamomum cassia, Allium tuberosum, Allium sativum, Rosa multiflora (Rosa multiflora fruit), Achillea, Rosa multiflora, Amygdalus davidiana, Cocos nucifera, Ananas comosus fruit, Ocimum basilicum, Nelumbo nucifera (Nelumbo nucifera fruit/seed), Petroselium crispum, Mentha arvensis var. piperascens (Mentha arvensis var. piperascens leaf), Coix lacryma-jobi var. frumentacea (Coix lacryma-jobi var. frumentacea seed), Musa acuminata, Origanum vulgare, Vanilla planifolia beans, Carica papaya, Capsicum annuum ‘grossum’, Hamamelis virginiana (Hamamelis virginiana leaf), Rosa, Belamcanda chinensis, Rabdosia japonica (Rabdosia japonica leaf), Trapa japonica (Trapa japonica fruit), Chamaecyparis obtusa, Ricinus communis, Helianthus annus, Capsicum annuum, Eriobotrya japonica (Eriobotrya japonica leaf), Areca catechu (Areca catechu peel/nut), Petasites japonicus, Tussilago farfara (Tussilago farfara bud/leaf), Vitis vinifera fruit or Vitis vinifera peel/seed/leaf, Fagus crenata, Cordyceps sinensis, Periandra mediterranea, Rubus fruticosus, Prunus fruit, Vaccinium corymbosum (Vaccinium myrtillus), Prunus domestica, Luffa cylindrica, Carthamus tinctorius, berry fruits, Bergenia ligulata (Bergenia ligulata root), Spinacia oleracea, Physalis alkekengi var. franchetii (Physalis alkekengi var. franchetii root), Tilia miqueliana, Paeonia suffruticosa (Paeonia suffruticosa root bark), Humulus lupulus, Simmondsia chinensis, Grifola frondosa, Ephedra sinica, Macadamia integrifolia nut, Morus alba (Morus alba root bark/leaf), Origanum majorana (Origanum majorana leaf), Morus alba fruit, Cydonia oblonga, Mangifera indica, Garcinia mangostana, Citrus reticulata fruit, Ganoderma lucidum, Mentha spicata, Acacia dealbata, Zingiber mioga, Synsepalum dulcificum fruit, Sapindus mukorossi, Lithospermum erythrorhizon (Lithospermum erythrorhizon root), Peruvian Zea mays, Melia azadirachta (Melia azadirachta leaf), Melissa officinalis, Melilotus officinalis, Cucumis melo fruit, Prunus persica leaf/fruit/kernel, Vigna radiata, Prunus cerasus var. austera fruit, Corchorus olitorius, Rodgersia podophylla (Centaurea cyanus), Monarda fistulosa, Artemisia montana, Eucalyptus globulus, Saxifraga stolonifera, Citrus junos fruit, Lilium lancifolium, Rubus idaeus subsp. idaeus, Artemisia princeps (Artemisia princeps leaf), Citrus aurantifolia fruit, Secale cereale, Rubus idaeus leaf/fruit, Lavandula dentata, Camellia sinensis leaf (green tea leaf), Malus domestica fruit, Gentiana scabra var. buergeri, Citrus limon fruit, Cymbopogon citratus, Forsythia suspensa, Astragalus sinicus, Rosmarinus officinalis, Rosa canina, Wasabia japonica, and Sanguisorba officinalis.

Other beautifying components can also be added. Examples include Saccharomyces cerevisiae, hydrolyzed conchiolin, hydrolyzed hyaluronic acid, hydroxypropyltrimonium hyaluronate, and (C12-C13)alkyl glyceryl hydrolyzed hyaluronate.

Properties of these active ingredients are considered in deciding in which one of the phases (inner aqueous phase, oil phase, and outer aqueous phase) these active ingredients are to be contained.

The component content of each phase relative to the total amount of the W/O/W emulsion can be determined as appropriate depending on various factors such as the types of an active ingredient(s) and a surfactant, and the type and amount of a solid oil component.

In terms of emulsion stability, the weight ratio of the inner aqueous phase to the oil phase, i.e., inner aqueous phase/oil phase, is preferably 10/90 to 90/10, more preferably 20/80 to 80/20, still more preferably 25/75 to 75/25. The proportion of the oil phase per 100 parts by weight of the inner aqueous phase is preferably 10 to 500 parts by weight, more preferably 20 to 300 parts by weight, still more preferably 50 to 200 parts by weight. The proportion of the outer aqueous phase per 100 parts by weight of the inner aqueous phase is preferably 50 to 2000 parts by weight, more preferably 100 to 1500 parts by weight, still more preferably 200 to 1000 parts by weight.

The W/O/W emulsion of the present invention may be in any form as long as it has temporal stability. For example, the average particle size of inner aqueous phase droplets may be 0.001 to 100 μm, preferably 0.01 to 100 μm, more preferably 0.1 to 100 μm; the average particle size of the W/O emulsion may be, for example 0.01 to 100 μm, preferably 0.1 to 100 μm, more preferably 0.5 to 100 μm, still more preferably 1 to 50 μm.

The viscosity of the W/O/W emulsion of the present invention at 25° C. is preferably 500 to 50,000 mPa·s, more preferably 1,000 to 40,000 mPa·s, still more preferably 2,000 to 30,000 mPa·s. Here, the viscosity is measured with a BM-type viscometer (available from Toki Sangyo Co., Ltd.; measurement conditions: rotor No. 4 at 12 rpm for 1 min.).

The W/O/W emulsion of the present invention can be used for various applications to which a W/O/W emulsion is applicable. In one embodiment, the W/O/W emulsion of the present invention can be used as an agent for external use (composition for external use). In one embodiment, the W/O/W emulsion of the present invention can be used as an oral composition (e.g., a food or beverage, an oral pharmaceutical, an oral quasi-pharmaceutical, etc.). In one embodiment, the W/O/W emulsion of the present invention can be suitably used as an agent for external use.

The agent for external use is applicable to the body surface such as skin and mucous membrane. Examples include cosmetics, quasi-pharmaceuticals for external use, pharmaceuticals for external use, cleaning agents, and perfumery. The agent for external use is preferably a skin agent for external use, for example.

The W/O/W emulsion of the present invention can be suitably used as a skin agent for external use, for example. The term “skin agent for external use” as used in the present invention refers to an agent that can be administered to the skin and represents a generic term for cosmetics including creams, serums, milky lotions, toners, and facial beauty masks as well as quasi-pharmaceuticals, pharmaceuticals, cleaning agents, perfumery, and the like. The skin agent for external use may be in any form but is preferably a cream or a milky lotion. The skin agent for external use of the present invention may be used in any known application to which emulsions are applicable. The skin agent for external use is preferably a cosmetic, more preferably a cream or a milky lotion.

### (Method of Producing W/O/W Emulsion)

The W/O/W emulsion of the present invention may be produced by any known method, but the following production method can produce a W/O/W emulsion that is particularly excellent in temporal stability.

For example, the W/O/W emulsion can be produced by a step of mixing and emulsifying an inner aqueous phase containing inositol and an oil phase to prepare a W/O emulsion; and a step of mixing and emulsifying the prepared W/O emulsion and an outer aqueous phase.

The present invention also encompasses a method of producing a W/O/W emulsion, including the step of preparing a W/O emulsion and the step of mixing and emulsifying the prepared W/O emulsion and an outer aqueous phase.

In the production method of the present invention, preferred embodiments of the inner aqueous phase, the oil phase, and the outer aqueous phase are as described above. For example, preferably, a solid oil component and a silicone surfactant are contained in the oil phase. Preferably, a water-soluble polymer is contained in the outer aqueous phase.

Preferably, the production of the W/O/W emulsion includes the following two steps:
(1) a step of mixing and emulsifying an inner aqueous phase containing inositol and an oil phase containing a solid oil component and a silicone surfactant to prepare a W/O emulsion; and
(2) a step of mixing and emulsifying the prepared W/O emulsion and an outer aqueous phase containing a water-soluble polymer.

Specifically, the inner aqueous phase containing inositol is added to the oil phase, followed by emulsification at an emulsification temperature of preferably 50° C. to 100° C., more preferably 60° C. to 95° C., still more preferably 65° C. to 85° C., whereby a W/O emulsion is prepared. Here, preferably, the inner aqueous phase and the oil phase are mixed at a weight ratio (inner aqueous phase/oil phase) of preferably 10/90 to 90/10, more preferably 20/80 to 80/20, still more preferably 25/75 to

Another optional component (e.g., metal salt) that may be optionally used may be contained in the inner aqueous phase. Usually, a liquid oil component is contained in the oil phase. The above-described optional component (e.g., oil-soluble active ingredient) may be contained in the oil phase.

Next, the W/O emulsion is added to the outer aqueous phase, followed by emulsification at an emulsification temperature of preferably 70° C. or lower, more preferably 0° C. to 60° C., still more preferably 10° C. to 50° C., whereby a W/O/W emulsion is prepared.

An acrylic acid/alkyl methacrylate copolymer may be contained in the outer aqueous phase. Preferably, inositol is absent in the outer aqueous phase.

Preferably, the mixing amount of each component is the amount at which the component content of the W/O/W emulsion is in the above-described range.

The numerical range defined by the lower limit and the upper limit herein, i.e., “the lower limit to the upper limit”, includes the lower limit and the upper limit. For example, the range defined by “1 to 2” means 1 or more and 2 or less, with 1 and 2 being inclusive. Herein, the range may be any combination of any upper limit and any lower limit.

### EXAMPLES

The present invention is described in further detail below with reference to Examples, which do not limit the present invention.

### Examples 1 to 4

A W/O/W emulsion containing inositol was prepared.

First, the components were mixed at a mixing ratio (wt %) shown in Table 1 to prepare an oil phase, an inner aqueous phase, and an outer aqueous phase. Next, the inner aqueous phase was fed into the oil phase at a temperature of 80° C., whereby a W/O emulsion was prepared. In preparing the W/O emulsion, the inner aqueous phase and the oil phase were mixed and emulsified. Subsequently, the W/O emulsion was fed into the outer aqueous phase at a temperature of 50° C. Thus, a W/O/W emulsion (W/O/W emulsion formulation) of each of Examples 1 to 4 was obtained. More specifically, the W/O emulsion and the outer aqueous phase were mixed and emulsified to obtain the W/O/W emulsion.

When visually observed, each W/O/W emulsion obtained had a milky lotion-like appearance. Optical microscope observation confirmed that each W/O/W emulsion obtained was one composed of an inner aqueous phase, an oil phase, and an outer aqueous phase.

The W/O/W emulsions of Examples 1 to 3 are W/O/W emulsions each containing inositol in the inner aqueous phase. The W/O/W emulsion of Example 4 is a W/O/W emulsion containing inositol in both the inner aqueous phase and the outer aqueous phase.

The values in Table 1 are the mixing amounts (wt %) of the components in the W/O/W emulsion (100 wt %). Inositol used was myo-inositol (available from Tsuno Rice Fine Chemicals Co., Ltd.).

### Comparative Example 1

A W/O/W emulsion (W/O/W emulsion formulation) was prepared by the same method as in Examples 1, except that the components were added in amounts shown in Table 1. The resulting W/O/W emulsion had a milky lotion-like appearance (visually observed).

### Reference Example 1

A W/O/W emulsion (W/O/W emulsion formulation) was prepared by the same method as in Example 1, except that the components were added in amounts shown in Table 1. The resulting W/O/W emulsion had a milky lotion-like appearance (visually observed).

The W/O/W emulsion prepared in Comparative Example 1 is a W/O/W emulsion containing inositol in the outer aqueous phase. The W/O/W emulsion prepared in Reference Example 1 is a W/O/W emulsion containing no inositol. Optical microscope observation in Comparative Example 1 and Reference Example 1 also confirmed that each W/O/W emulsion obtained was one composed of an inner aqueous phase, an oil phase, and an outer aqueous phase.

TABLE 1

| | | | | | | Comparative | Reference |
|---|---|---|---|---|---|---|---|
| | Component name | Example 1 | Example 2 | Example 3 | Example 4 | Example 1 | Example 1 |
| Inner aqueous | Purified water | 10 | 10 | 10 | 10 | 10 | 10 |
| phase | Magnesium sulfate | 0.5 | 0.5 | 0.5 | 0.5 | 0.5 | 0.5 |
| | Inositol | 0.1 | 0.3 | 0.5 | 0.5 | 0 | 0 |
| Oil phase | Cetyl PEG/PPG-10/1 dimethicone | 2 | 2 | 2 | 2 | 2 | 2 |
| | Squalane | 8 | 8 | 8 | 8 | 8 | 8 |
| | Microcrystalline wax | 0.9 | 0.9 | 0.9 | 0.9 | 0.9 | 0.9 |
| Outer | Methyl p-oxybenzoate | 0.1 | 0.1 | 0.1 | 0.1 | 0.1 | 0.1 |
| aqueous | 1,3-Butylene glycol | 13 | 13 | 13 | 13 | 13 | 13 |
| phase | Concentrated glycerol | 2 | 2 | 2 | 2 | 2 | 2 |
| | Purified water | 60.1 | 59.9 | 59.7 | 59.2 | 59.7 | 60.2 |
| | Inositol | 0 | 0 | 0 | 0.5 | 0.5 | 0 |
| | Acrylic acid/alkyl methacrylate copolymer | 0.1 | 0.1 | 0.1 | 0.1 | 0.1 | 0.1 |
| | Polyoxyethylene sorbitan monooleate (20E.O.) | 2 | 2 | 2 | 2 | 2 | 2 |
| | Triethanolamine | 0.2 | 0.2 | 0.2 | 0.2 | 0.2 | 0.2 |
| | Ethanol | 1 | 1 | 1 | 1 | 1 | 1 |

| | | | | | | |
|---|---|---|---|---|---|---|
| Total (wt %) | 100 | 100 | 100 | 100 | 100 | 100 |

The components shown in Table 1 are as follows. Cetyl PEG/PPG-10/1 dimethicone: available from Evonik Operations GmbH, HLB 5.0

Squalane: available from Nikko Chemicals Co., Ltd.
Microcrystalline wax: available from Nikko Rica Corporation Acrylic acid/alkyl methacrylate copolymer: available from Lubrizol Advanced Materials, Inc.
Polyoxyethylene sorbitan monooleate (20E.O.): available from Kao Corporation

### <Evaluation of W/O/W Emulsion>

The temporal stability of the W/O/W emulsion prepared in each of Examples, Comparative Example, and Reference Example was evaluated.

The emulsion formulations prepared were stored under acceleration conditions at 50° C., and the appearance of each formulation was visually checked at the time of production (immediately after production), after storage for one month, and after storage for two months to see if separation has occurred. The appearance was evaluated based on the following criteria. Table 2 shows the results.

### (Evaluation Criteria)

o: No separation is observed.
Δ: Slight separation is observed.
x: Separation is observed.

TABLE 2

| | | | | | Comparative | Reference |
|---|---|---|---|---|---|---|
| | Example 1 | Example 2 | Example 3 | Example 4 | Example 1 | Example 1 |
| At the time of production | ∘ | ∘ | ∘ | ∘ | ∘ | ∘ |
| One month later | ∘ | ∘ | ∘ | Δ | x | ∘ |
| Two months later | ∘ | ∘ | ∘ | x | x | ∘ |

The W/O/W emulsion of Comparative Example 1, which contained inositol in the outer aqueous phase, was found to be separated after storage of one month, indicating a low temporal stability.

In contrast, the W/O/W emulsion of each of Examples 1 to 4, which contained inositol in the inner aqueous phase, had a high temporal stability. In particular, the W/O/W emulsions of Examples 1 to 3 each had an excellent temporal stability without separation after storage and without change in the appearance. The W/O/W emulsion of Example 4 was found to have an improved temporal stability as compared to the W/O/W emulsion of Comparative Example 1.

### Examples 5 and 6

A W/O/W emulsion (W/O/W emulsion formulation) containing inositol in the inner aqueous phase was prepared by the same method as in Example 1, except that the components were added in amounts shown in Table 3. The resulting W/O/W emulsion had a milky lotion-like appearance (visually observed). Optical microscope observation confirmed that each W/O/W emulsion obtained was one composed of an inner aqueous phase, an oil phase, and an outer aqueous phase. The values in Table 3 are the mixing amounts (wt %) of the components in the W/O/W emulsion (100 wt %). The same components were used as in Example 1.

The W/O/W emulsions obtained in Examples 5 and 6 were stored at room temperature for six months, and the appearance was visually evaluated. No change in the appearance was found for six months in the W/O/W emulsions obtained in Examples 5 and 6, and no separation occurred. The temporal stability of these W/O/W emulsions was confirmed. The W/O/W emulsions of the present invention were confirmed to be suitable for use as a milky lotion or cream, for example.

TABLE 3

| | Component name | Example 5 | Example 6 |
|---|---|---|---|
| Inner | Purified water | 10 | 10 |
| aqueous | Magnesium sulfate | 0.5 | 0.5 |
| phase | Inositol | 0.001 | 1.4 |
| Oil phase | Cetyl PEG/PPG-10/1 | 2 | 2 |
| | dimethicone | | |
| | Squalane | 8 | 8 |
| | Microcrystalline wax | 0.5 | 0.6 |
| Outer | Methyl p-oxybenzoate | 0.1 | 0.1 |
| aqueous | 1,3-Butylene glycol | 13 | 13 |
| phase | Concentrated glycerol | 2 | 2 |
| | Purified water | 60.599 | 59.1 |
| | Acrylic acid/alkyl | 0.1 | 0.1 |
| | methacrylate copolymer | | |
| | Polyoxyethylene sorbitan | 2 | 2 |
| | monooleate (20E.O.) | | |
| | Triethanolamine | 0.2 | 0.2 |
| | Ethanol | 1 | 1 |

| | | |
|---|---|---|
| Total (wt %) | 100 | 100 |

The results show that the W/O/W emulsion can be stored stably when inositol is added to the inner aqueous phase of the W/O/W emulsion.

### INDUSTRIAL APPLICABILITY

The present invention can provide a W/O/W emulsion containing inositol and having high temporal stability and a method of producing the W/O/W emulsion.

1. A W/O/W emulsion containing an inner aqueous phase, an oil phase, and an outer aqueous phase, the W/O/W emulsion comprising:
inositol in the inner aqueous phase.

2. The W/O/W emulsion according to claim 1,
wherein the W/O/W emulsion contains no inositol in the outer aqueous phase.

3. The W/O/W emulsion according to claim 1,
wherein the W/O/W emulsion has an inositol content of 0.01 to 1.4 wt %.

4. The W/O/W emulsion according to claim 1,
wherein the W/O/W emulsion contains a solid oil component and a silicone surfactant in the oil phase, and a water-soluble polymer in the outer aqueous phase.

5. The W/O/W emulsion according to claim 4,
wherein the silicone surfactant has an HLB value of 7 or less.

6. The W/O/W emulsion according to claim 4,
wherein the silicone surfactant is a silicone surfactant having a polyether group and an alkyl group.

7. The W/O/W emulsion according to claim 6,
wherein the silicone surfactant having a polyether group and an alkyl group is cetyl PEG/PPG-10/1 dimethicone or lauryl PEG-9 polydimethylsiloxyethyl dimethicone.

8. The W/O/W emulsion according to claim 4,
wherein the W/O/W emulsion has a solid oil component content of 0.5 wt % or more.

9. The W/O/W emulsion according to claim 4,
wherein the solid oil component is a wax.

10. The W/O/W emulsion according to claim 4,
wherein the water-soluble polymer is an acrylic acid/alkyl methacrylate copolymer.

11. The W/O/W emulsion according to claim 1, further comprising a metal salt in the inner aqueous phase.

12. The W/O/W emulsion according to claim 1,
wherein a weight ratio of the inner aqueous phase to the oil phase is 10/90 to 90/10.

13. The W/O/W emulsion according to claim 1,
wherein the W/O/W emulsion is an agent for external use.

14. The W/O/W emulsion according to claim 1,
wherein the W/O/W emulsion is a skin agent for external use.

15. The W/O/W emulsion according to claim 1,
wherein the W/O/W emulsion is a cream or a milky lotion.

16. A method of producing a W/O/W emulsion, comprising:
a step of mixing and emulsifying an inner aqueous phase containing inositol and an oil phase to prepare a W/O emulsion; and
a step of mixing and emulsifying the prepared W/O emulsion and an outer aqueous phase.

17. The method of producing a W/O/W emulsion according to claim 16,
wherein the outer aqueous phase contains no inositol.

18. The method of producing a W/O/W emulsion according to claim 16,
wherein the oil phase contains a solid oil component and a silicone surfactant, and
the outer aqueous phase contains a water-soluble polymer.

19. The method of producing a W/O/W emulsion according to claim 16,
wherein the method comprises mixing the inner aqueous phase and the oil phase at a weight ratio of 10/90 to 90/10.
